(19) United States
(12) Patent Application Publication
(10) Pub. No.: US 2026/0158025 A1
(43) Pub. Date: Jun. 11, 2026

(54) COMPOUNDS AND COMBINATIONS THEREOF FOR TREATING NEUROLOGICAL AND PSYCHIATRIC CONDITIONS

(71) Applicant: ANTECIP BIOVENTURES II LLC, New York, NY (US)

(72) Inventor: Herriot Tabuteau, New York, NY (US)

(21) Appl. No.: 19/387,351

(22) Filed: Nov. 12, 2025

Related U.S. Application Data
(63) Continuation of application No. 19/059,105, filed on Feb. 20, 2025.
(60) Provisional application No. 63/557,860, filed on Feb. 26, 2024.

(51) Int. Cl. A61K 31/485; A61K 9/20; A61K 9/24; A61K 31/137; A61P 25/00

(52) U.S. Cl. CPC A61K 31/485; A61K 9/2009; A61K 9/2013; A61K 9/2027; A61K 9/2054; A61K 9/209; A61K 31/137; A61P 25/00

(57) ABSTRACT

This disclosure relates to a method of treating a neurological disorder, comprising administering a bupropion, or a combination of a bupropion and a dextromethorphan, to a human being in need thereof, monitoring the human being for Brugada pattern/syndrome, drug reaction with eosinophilia and systemic symptoms, or acute generalized exanthematous pustulosis, and reducing or discontinuing use of the bupropion, or the combination of the bupropion and the dextromethorphan, if the human being develops Brugada pattern/syndrome, subcutaneous tissue disorder, drug reaction with eosinophilia and systemic symptoms (DRESS), or acute generalized exanthematous pustulosis.

### CROSS-REFERENCE TO RELATED PATENT APPLICATION

This application is a continuation of U.S. patent application Ser. No. 19/059,105, filed Feb. 20, 2025; which claims benefit of U.S. Provisional Pat. App. No. 63/557,860, filed Feb. 26, 2024; both of which are incorporated by reference by their entirety.

### SUMMARY

This disclosure relates to administration of a bupropion. Some embodiments related to a combination of a bupropion and a dextromethorphan for treating a neurological disorder.

Some embodiments include a method of treating a neurological disorder, comprising administering a bupropion or a combination of a bupropion and a dextromethorphan to a human being in need thereof, monitoring the human being for Brugada pattern/syndrome, subcutaneous tissue disorder, drug reaction with eosinophilia and systemic symptoms (DRESS), or acute generalized exanthematous pustulosis, and reducing or discontinuing use of the bupropion or a combination of a bupropion and a dextromethorphan if the human being develops Brugada pattern/syndrome, subcutaneous tissue disorder, drug reaction with eosinophilia and systemic symptoms, or acute generalized exanthematous pustulosis.

### BRIEF DESCRIPTION OF THE DRAWINGS

FIG. 1 depicts the effects of renal impairment, hepatic impairment, and CYP2D6 poor metabolizer status on the pharmacokinetics of a tablet containing 45 mg of dextromethorphan hydrobromide and 105 mg of bupropion hydrochloride.

FIG. 2 depicts the change from baseline in MADRS total score by week for human patients receiving a tablet containing 45 mg of dextromethorphan hydrobromide and 105 mg of bupropion hydrochloride twice per day.

### DETAILED DESCRIPTION

As mentioned above, this disclosure relates to a method of treating a neurological disorder, comprising administering a bupropion to a human being in need thereof, monitoring the human being for Brugada pattern/syndrome, subcutaneous tissue disorder, drug reaction with eosinophilia and systemic symptoms, or acute generalized exanthematous pustulosis, and reducing or discontinuing use of the bupropion if the human being develops Brugada pattern/syndrome, subcutaneous tissue disorder, drug reaction with eosinophilia and systemic symptoms (DRESS), or acute generalized exanthematous pustulosis.

In some embodiments, about 5-100 mg, about 100-150 mg, about 150-200 mg, about 200-250 mg, about 250-300 mg, or about 300-450 mg of bupropion is administered per day to the human being in need of treatment for a neurological disorder, and the human being is monitored for Brugada pattern/syndrome, subcutaneous tissue disorder, drug reaction with eosinophilia and systemic symptoms, or acute generalized exanthematous pustulosis, and reducing or discontinuing use of the bupropion if the human being develops Brugada pattern/syndrome, subcutaneous tissue disorder, drug reaction with eosinophilia and systemic symptoms (DRESS), or acute generalized exanthematous pustulosis.

Some embodiments relate to a method of treating a neurological disorder, comprising administering a combination of a bupropion and a dextromethorphan to a human being in need thereof, monitoring the human being for Brugada pattern/syndrome, subcutaneous tissue disorder, drug reaction with eosinophilia and systemic symptoms, or acute generalized exanthematous pustulosis, and reducing or discontinuing use of the combination of the bupropion and the dextromethorphan if the human being develops Brugada pattern/syndrome, subcutaneous tissue disorder, drug reaction with eosinophilia and systemic symptoms (DRESS), or acute generalized exanthematous pustulosis.

In some embodiments, a combination of a bupropion in an amount such as about 5-100 mg, about 100-150 mg, about 150-200 mg, about 200-250 mg, about 250-300 mg, or about 300-450 mg of the bupropion per day, and a dextromethorphan in an amount such as about 30-50 mg, about 50-100 mg, or about 100-150 mg of the dextromethorphan per day, are administered to the human being in need of treatment for a neurological disorder, and the human being is monitored for Brugada pattern/syndrome, subcutaneous tissue disorder, drug reaction with eosinophilia and systemic symptoms, or acute generalized exanthematous pustulosis, and reducing or discontinuing use of the bupropion, or the combination of bupropion and dextromethorphan, if the human being develops Brugada pattern/syndrome, subcutaneous tissue disorder, drug reaction with eosinophilia and systemic symptoms (DRESS), or acute generalized exanthematous pustulosis.

In some embodiments, a combination of: 1) about 100-110 mg, about 104-106 mg, or about 105 mg of bupropion hydrochloride, or a molar equivalent amount of the free base form or another salt form of dextromethorphan; and 2) about 40-50 mg, about 44-46 mg, or about 45 mg of dextromethorphan hydrobromide, or a molar equivalent amount of the free base form or another salt form of dextromethorphan, are administered once or twice a day to the human being in need of treatment for a neurological disorder. This combination is referred to for convenience herein as the “subject combination.” In every instance where the subject combination is referred to herein, the combination of 105 mg of bupropion hydrochloride and 45 mg of dextromethorphan hydrobromide is specifically contemplated.

It has been found that treatment with bupropion may result in a patient developing a cardiovascular adverse event, such as complete atrioventricular block, extrasystoles, hypotension, hypertension (in some cases severe), phlebitis, pulmonary embolism, or Brugada pattern/syndrome. Patients being treated with a bupropion or a combination of a bupropion and a dextromethorphan, including the subject combination, should be monitored for these adverse events.

It has been found that treatment with bupropion may result in a patient developing Skin and subcutaneous tissue disorders, such as alopecia, angioedema, exfoliative dermatitis, hirsutism, Stevens-Johnson syndrome, acute generalized exanthematous pustulosis, and drug reaction with eosinophilia and systemic symptoms (DRESS). Patients being treated with a bupropion or a combination of a bupropion and a dextromethorphan, including the subject combination, should be monitored for these disorders.

In some embodiments, the patient is monitored for potentially developing a subcutaneous tissue disorder, and the bupropion, or the combination of a bupropion and a dextromethorphan, including the subject combination, is discontinued if the patient develops this disorder.

In some embodiments, the patient is monitored for potentially developing DRESS, and the bupropion, or the combination of a bupropion and a dextromethorphan, including the subject combination, is discontinued if the patient develops this disorder.

In some embodiments, the patient is monitored for potentially developing Brugada pattern/syndrome, and the bupropion, or the combination of a bupropion and a dextromethorphan, including the subject combination, is discontinued if the patient develops this adverse event.

In some embodiments, the patient is monitored for potentially developing acute generalized exanthematous pustulosis, and the bupropion, or the combination ofa bupropion and a dextromethorphan, including the subject combination, is discontinued if the patient develops this disorder.

In some embodiments, if the human being develops Brugada pattern/syndrome, subcutaneous tissue disorder, drug reaction with eosinophilia and systemic symptoms (DRESS), or acute generalized exanthematous pustulosis, the bupropion treatment is discontinued.

In some embodiments, if the human being develops Brugada pattern/syndrome, subcutaneous tissue disorder, drug reaction with eosinophilia and systemic symptoms (DRESS), or acute generalized exanthematous pustulosis, the bupropion treatment is reduced. For example, about 105 mg or less per day of bupropion, or a pharmaceutically acceptable salt thereof, is administered.

Dextromethorphan hydrobromide is an uncompetitive NMDA receptor antagonist and a sigma-1 receptor agonist.

The chemical name of dextromethorphan hydrobromide is morphinan, 3-methoxy-17-methyl-, (9α, 13α, 14α), hydrobromide monohydrate. Dextromethorphan hydrobromide has the empirical formula C₁₈H₂₅NO·HBr·H₂O and a molecular weight of 370.33. The structural formula is:

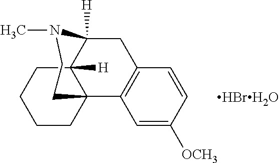 [SMILES: COc1ccc2c(c1)[C@]13CCCC[C@@H]1[C@H](C2)N(C)CC3]

Dextromethorphan hydrobromide powder is white or almost white, crystalline, and sparingly soluble in water.

Bupropion hydrochloride is an aminoketone and CYP450 2D6 inhibitor.

The chemical name of bupropion hydrochloride is: (±)-1-(3-chlorophenyl)-2-[(1,1-dimethylethyl)amino]-1-propanone hydrochloride. Bupropion hydrochloride has the empirical formula C₁₃H₁₈ClNO·HCl and a molecular weight of 276.2. The structural formula is:

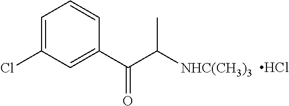

Bupropion hydrochloride powder is white and highly soluble in water.

The subject combination may be contained in an oral dosage form, including a tablet, such as an extended-release tablet. In some embodiments, the subject combination is contained in a dosage form for oral administration and is available as round bilayer tablets.

In some embodiments, each tablet containing the subject combination contains 45 mg of dextromethorphan hydrobromide in an immediate-release formulation. In some embodiments, each tablet of the subject combination contains 105 mg of bupropion hydrochloride in an extended-release formulation. In some embodiments, each tablet of the subject combination contains 45 mg of dextromethorphan hydrobromide in an immediate-release formulation and 105 mg of bupropion hydrochloride in an extended-release formulation.

In some embodiments, a tablet containing the subject combination contains L-cysteine hydrochloride monohydrate. In some embodiments, a tablet containing the subject combination contains carbomer homopolymer. In some embodiments, a tablet containing the subject combination contains microcrystalline cellulose. In some embodiments, a tablet containing the subject combination contains colloidal silicon dioxide. In some embodiments, a tablet containing the subject combination contains crospovidone. In some embodiments, a tablet containing the subject combination contains stearic acid. In some embodiments, a tablet containing the subject combination contains magnesium stearate.

In some embodiments, a tablet containing the subject combination contains the following inactive ingredients: L-cysteine hydrochloride monohydrate, carbomer homopolymer, microcrystalline cellulose, colloidal silicon dioxide, crospovidone, stearic acid, and magnesium stearate.

In some embodiments, the starting dosage of the subject combination is 45 mg of dextromethorphan hydrobromide and 105 mg of bupropion hydrochloride in one tablet that is administered once daily in the morning. In some embodiments, after 3 days, the dosage is increased to one tablet (or one dosage form containing 45 mg of dextromethorphan hydrobromide and 105 mg of bupropion hydrochloride) twice daily, e.g., given at least 8 hours apart. In some embodiments, no more than two doses containing 45 mg of dextromethorphan hydrobromide and 105 mg of bupropion hydrochloride are administered in the same day.

No dose adjustment is recommended in patients with mild (Child-Pugh A) or moderate hepatic impairment (Child-Pugh B) as compared with the dose recommended to a patient with normal hepatic function.

The pharmacokinetics of the combination of 45 mg of dextromethorphan hydrobromide and 105 mg of bupropion hydrochloride have not been evaluated in patients with severe hepatic impairment (Child-Pugh C). Use of combination of 45 mg of dextromethorphan hydrobromide and 105 mg of bupropion hydrochloride is not recommended, or should be avoided, in patients with severe hepatic impairment.

The subject combination may be administered orally with or without food. In some embodiments, the tablets are swallowed whole, and not crushed, divided, or chewed.

Patients having renal impairment may require special dosing.

In some embodiments, the renal impairment status of the subject may be determined by measuring the “estimated glomerular filtration rate” or “eGFR” of the individual. The eGFR in mL/min/1.73 m² is calculated by the Modification of Diet in Renal Disease [MDRD] equation:

$$( eGFR in mL / min ) / 1.73 {m}^{2} = 175 \times {( serum creatinine in mg / dL )}^{- 1.154} \times {Age}^{- 0.203} \times 0.742 ( if female ) \times 1.212 ( if black ) .$$

Further details regarding the calculation of the eGFR may be found in, e.g., Levey A S, Coresh J, Greene T, Marsh J, Stevens L A, Kusek J W, Van Lente F: Chronic Kidney Disease Epidemiology Collaboration. Expressing the Modification of Diet in Renal Disease Study Equation for Estimating Glomerular Filtration Rate with Standardized Serum Creatinine Values. Ann Intern Med. 2009; 150(9):604-12.

Renal impairment status based on Food and Drug Administration (FDA) guidance is as follows.
Normal: eGFR≥90 mL/min/1.73 m²
Mild: eGFR 60-89 mL/min/1.73 m2 (i.e., ≥60 to <90)
Moderate: eGFR 30-59 mL/min/1.73 m² (i.e., ≥30 to <60)
Severe: eGFR 15-29 mL/min/1.73 m² (i.e., ≥15 to <30) and not on hemodialysis
End-stage renal disease (ESRD): eGFR<15 mL/min/1.73 m² and not on hemodialysis or on hemodialysis
   See, Guidance for Industry Pharmacokinetics in Patients with Impaired Renal Function—Study Design, Data Analysis and Impact on Dosing and Labeling. U.S. Department of Health and Human Services Food and Drug Administration Center for Drug Evaluation and Research (CDER) Center for Biologics Evaluation and Research (CBER) February 2010. As used herein, a “renally impaired subject” may have mild, moderate, or severe renal impairment, or may have ESRD.

In some embodiments, the recommended dosage of the subject combination for patients with moderate renal impairment (estimated glomerular filtration rate (eGFR) or glomerular filtration rate (GFR) of 30 to 59 mL/minute/1.73 m²) is a daily dose of 45 mg of dextromethorphan hydrobromide and 105 mg of bupropion hydrochloride, or a molar equivalent amount of another form of dextromethorphan and/or bupropion, such as administration of one tablet (or one dosage form containing 45 mg of dextromethorphan hydrobromide and 105 mg of bupropion hydrochloride) once daily, such as one tablet or other oral dosage form daily in the morning, or twice daily oral administration of a combination of 52.5 mg of bupropion hydrochloride and about 22.5 mg of dextromethorphan hydrobromide. In some embodiments, the patients are monitored for adverse reactions potentially attributable to dextromethorphan, such as somnolence and dizziness.

In some embodiments, the subject combination is in a dosage form, such as a tablet, which further comprises: colloidal silicon dioxide, crospovidone, glyceryl monocaprylocaprate, L-cysteine hydrochloride monohydrate, magnesium stearate, microcrystalline cellulose, polyvinyl alcohol, red iron oxide, sodium lauryl sulfate, stearic acid, talc, titanium dioxide, or yellow iron oxide, or a combination thereof.

Moderate renal impairment may be determined by an assay, such as an assay that determines creatinine levels, on a biological sample from the patient, such as a blood sample. Serum creatinine levels from a blood sample can be used to estimate GFR.

Patients who are concomitantly using the subject combination with strong CYP2D6 inhibitors may require special dosing. Concomitant use of the subject combination with a strong CYP2D6 inhibitor increases plasma concentrations of dextromethorphan. In some embodiments, the recommended dosage of the subject combination when coadministered with a strong CYP2D6 inhibitor is one tablet (or one dosage form containing 45 mg of dextromethorphan hydrobromide and 105 mg of bupropion hydrochloride) once daily, such as one tablet or other oral dosage form daily in the morning. In some embodiments, the patients are monitored for adverse reactions potentially attributable to dextromethorphan, such as somnolence and dizziness.

Strong CYP2D6 inhibitors include, but are not limited to, compounds such as fluoxetine, propafenone, quinidine, and paroxetine.

Patients who are known CYP2D6 poor metabolizers (PMs) may require special dosing. In some embodiments, the recommended dosage for patients known to be poor CYP2D6 metabolizers is one tablet (or one dosage form containing 45 mg of dextromethorphan hydrobromide and 105 mg of bupropion hydrochloride) once daily, such as one tablet or other oral dosage form daily in the morning.

Special precautions may be required when switching a patient to or from a monoamine oxidase inhibitor (MAOI) antidepressant to the subject combination. In some embodiments, at least 14 days must elapse between discontinuation of an MAOI intended to treat depression and initiation of therapy with the subject combination. Conversely, in some embodiments, at least 14 days must be allowed after stopping the subject combination before starting an MAOI antidepressant.

There is data that reports the presence of bupropion and its metabolites in human milk. There is no data on the effects of bupropion or its metabolites on milk production. Limited data related to bupropion use in lactating patients have not identified a clear association of adverse reactions in the breastfed infant.

Neurotoxicity findings were observed in juvenile rats treated with a combination of dextromethorphan/quinidine on postnatal day (PND) 7, which corresponds to the third trimester of gestation through the first few months of life and may extend through the first three years of life in humans. It is not known whether dextromethorphan is present in human milk. There are no data on the effects of dextromethorphan on the breastfed infant or the effects on milk production. Because of the potential for neurotoxicity, in some embodiments, patients should be advised that breastfeeding is not recommended during treatment with a combination of dextromethorphan and bupropion and for 5 days following final dose.

In some embodiments, the human child of a patient receiving the subject combination is at risk of seizure. In some embodiments, the human child is about three years old or younger. In some embodiments, the human child of a patient receiving the subject combination is at risk of neurotoxicity.

For lactating human mothers, the combination of about 45 mg of dextromethorphan hydrobromide and about 105 mg of bupropion hydrochloride may be taken twice daily for about 1 week to about 6 weeks, about 6 weeks, or at least 6 weeks, before stopping the treatment at least 5 days before beginning breastfeeding the child.

In some embodiments, the human patient produces milk that is substantially free of dextromethorphan after 5 days from the final dose of the combination.

In some embodiments, the human patient produces milk that is substantially free of bupropion after 5 days from the final dose of the combination.

In some embodiments, the human child of a patient receiving the subject combination is not exposed to a detectable amount of dextromethorphan during breastfeeding.

In some embodiments, the human child of a patient receiving the subject combination is not exposed to a detectable amount of bupropion during breastfeeding.

In some embodiments, the subject combination is administered to the human patient twice daily until the human patient experiences a reduction from baseline of at least about 7, at least about 11, at least about 13, or at least about 15.9 in the human patient's total MADRS score. When the desired reduction in the MADRS score is reached, the human patient may stop the treatment, wait at least 5 day, and then begin breastfeeding her child.

In some embodiments, the human child is a breastfed infant.

In some embodiments, the human patient experiences a reduction from baseline in total MADRS score in the human patient for administering the subject combination that is more than the patient would experience if a placebo were administered. The human patient taking the subject combination may experience the greater reduction in MADRS score than taking a placebo at, for example, after 1 week of administration, after 2 weeks of administration, after 3 weeks of administration, after 4 weeks of administration, after 5 weeks of administration, after 6 weeks of administration, or at other times.

In the subject combination, bupropion inhibits the metabolism of dextromethorphan via CYP2D6. Dextromethorphan, when co-administered with bupropion, displays nonlinear pharmacokinetics at steady state, with greater than dose-proportional changes in AUC and Cₘₐₓ for varying doses of dextromethorphan (30 to 60 mg) and less than dose-proportional changes for varying doses of bupropion (75 to 150 mg).

Steady state plasma concentrations of dextromethorphan and bupropion when given as the subject combination are achieved within 8 days. The accumulation ratios for dextromethorphan at steady state are about 20 and about 32, respectively based on Cₘₐₓ and AUC₀₋₁₂. The accumulation ratios for bupropion at steady state are 1.1 and 1.5, respectively based on Cₘₐₓ and AUC₀₋₁₂.

After administration of the subject combination, the median Tₘₐₓ of dextromethorphan is about 3 hours and the median Tₘₐₓ of bupropion is about 2 hours. The Cₘₐₓ of hydroxybupropion metabolite occurs approximately 3 hours post-dose and is approximately 14 times the peak level of bupropion. The AUC₀₋₁₂ hydroxybupropion is about 19 times that of bupropion. The Cₘₐₓ of the erythrohydroxybupropion and threohydroxybupropion metabolites occurs approximately 4 hours post-dose and is approximately equal to and about 5 times that of bupropion, respectively. The AUC₀₋₁₂ values of erythrohydroxybupropion and threohydroxybupropion are about 1.2 and about 7 times that of bupropion, respectively.

The subject combination can be taken with or without food. Dextromethorphan Cₘₐₓ and AUC₀₋₁₂ were unchanged and decreased by 14%, respectively, and bupropion Cₘₐₓ and AUC₀₋₁₂ were increased by 3% and 6%, respectively, when the subject combination was administered with food.

The plasma protein binding of dextromethorphan is approximately 60-70% and bupropion is 84%. The extent of protein binding of the hydroxybupropion metabolite is similar to that for bupropion; whereas the extent of protein binding of the threohydroxybupropion metabolite is about half that seen with bupropion.

Following 8 days of administration of the subject combination in extensive metabolizers, the mean elimination half-life of dextromethorphan was increased approximately 3-fold to about 22 hours, as compared to dextromethorphan given without bupropion.

The mean elimination half-life of dextromethorphan and bupropion was 22 hours and 15 hours, respectively. The apparent elimination half-life of hydroxybupropion, erythrohydroxybupropion and threohydroxybupropion metabolites were approximately 35, 44 and 33 hours, respectively.

Esketamine is a non-competitive NMDA receptor antagonist indicated, in conjunction with an oral antidepressant, for the treatment of treatment-resistant depression in adults. Treatment of treatment-resistant depression carries a risk of dissociation. The label for esketamine states that because of the risks of sedation and dissociation, patients must be monitored for at least 2 hours at each treatment session, followed by an assessment to determine when the patient is considered clinically stable and ready to leave the healthcare setting.

Dissociation includes: delusional perception; depersonalization/derealization disorder; derealization; diplopia; dissociation; dysesthesia; feeling cold; feeling hot; feeling of body temperature change; hallucination; hallucination, auditory; hallucination, visual; hyperacusis; illusion; ocular discomfort; oral dysesthesia; paranesthesia; paranesthesia oral; pharyngeal paranesthesia; photophobia; time perception altered; tinnitus; vision blurred; visual impairment.

The subject combination is a combination of dextromethorphan, an uncompetitive N-methyl D-aspartate (NDMA) receptor antagonist and sigma-1 receptor agonist, and bupropion, an aminoketone and CYP450 2D6 inhibitor, indicated for the treatment of major depressive disorder (MDD) in adults. Unlike esketamine, the subject combination can be administered as a without dissociation or dissociative events. In some embodiments, the patient is not monitored for dissociation after the subject combination is administered.

The following information includes the kinds of dissociative and other effects that may be avoided with the combination of dextromethorphan and bupropion.

In clinical trials, 48% to 61% of esketamine-treated patients developed sedation based on the Modified Observer's Assessment of Alertness/Sedation scale (MOAA/S), and 0.3% to 0.4% of esketamine-treated patients experienced loss of consciousness (MOAA/S score of 0). For a combination of dextromethorphan and bupropion, NMDA receptor antagonism may be achieved without a significant risk of sedation.

Because of the possibility of delayed or prolonged sedation, patients receiving esketamine must be monitored by a healthcare provider for at least 2 hours at each treatment session, followed by an assessment to determine when the patient is considered clinically stable and ready to leave the healthcare setting. No such monitoring is required for the treatment of the subject combination of dextromethorphan and bupropion.

Esketamine-treated patients must be closely monitored for sedation with concomitant use of ESKETAMINE with CNS depressants. No such monitoring is required for the treatment of the subject combination of dextromethorphan and bupropion.

Esketamine is available only through a restricted program under a Risk Evaluation and Mitigation Strategy (REMS). No such restrictions are required for the subject combination of dextromethorphan and bupropion.

The most common psychological effects of esketamine were dissociative and/or perceptual changes (including distortion of time, space and illusions), derealization and depersonalization (61% to 84% of esketamine-treated patients developed dissociative or perceptual changes based on the Clinician-Administered Dissociative States Scale). Given its potential to induce dissociative effects, patients with psychosis must be carefully assessed before administering esketamine. No such assessment is required for the treatment of the subject combination of dextromethorphan and bupropion. Treatment with esketamine should be initiated only if the benefit outweighs the risk. No such restriction is required with the treatment of the subject combination of dextromethorphan and bupropion.

Because of the risks of dissociation, patients receiving esketamine must be monitored by a healthcare provider for at least 2 hours at each treatment session, followed by an assessment to determine when the patient is considered clinically stable and ready to leave the healthcare setting. No such monitoring, assessment, or restriction is required for the treatment of the subject combination of dextromethorphan and bupropion.

Dissociation includes: delusional perception (including distortion of space and time, and illusions); depersonalization/derealization disorder; derealization; diplopia; dissociation; dysesthesia; feeling cold; feeling hot; feeling of body temperature change; hallucination; hallucination, auditory; hallucination, visual; hallucinations, mixed; hyperacusis; illusion; ocular discomfort; oral dysesthesia; paresthesia; paresthesia oral; pharyngeal paresthesia; photophobia; time perception altered; tinnitus; vision blurred; and visual impairment.

Esketamine is a Schedule Ill controlled substance (CIII), and may be subject to abuse and diversion. The subject combination of dextromethorphan and bupropion is not a controlled substance. Each patient's risk for abuse or misuse must be assessed prior to prescribing and all patients receiving esketamine must be monitored for the development of these behaviors or conditions, including drug-seeking behavior, while on therapy. No such monitoring or assessment is required for the treatment of the subject combination of dextromethorphan and bupropion.

Esketamine is available only through a restricted program under a Risk Evaluation and Mitigation Strategy (REMS) and is called the esketamine REMS because of the risks of serious adverse outcomes from sedation, dissociation, and abuse and misuse. No such restrictions are required for the subject combination of dextromethorphan and bupropion.

Important requirements of the esketamine REMS include the following:
Healthcare settings must be certified in the program and ensure that esketamine is:
   Only dispensed and administered in healthcare settings.
   Patients treated in outpatient settings (e.g. medical offices and clinics) must be enrolled in the program.
   Administered by patients under the direct observation of a healthcare provider and that patients are monitored by a healthcare provider for at least 2 hours after administration of esketamine.
      None of these requirements apply to the subject combination of dextromethorphan and bupropion.

Esketamine was evaluated for safety in 262 adults for the treatment of depressive symptoms in adults with major depressive disorder (MDD) with acute suicidal ideation or behavior from two Phase 3 studies (Study 3 and Study 4) and one Phase 2 study.

Sedation was evaluated by adverse event reports and the Modified Observer's Assessment of Alertness/Sedation (MOAA/S). In the MOAA/S, a score of 5 means “responds readily to name spoken in normal tone;” a score of 0 means “no response after painful trapezius squeeze;” a score of 4 means “lethargic response to name spoken in normal tone;” a score of 3 means “responds only after name is spoken loudly and/or repeatedly;” a score of 2 means “responds only after mild prodding or shaking;” and a score of 1 means “dose not respond to mild prodding or shaking.” Any decrease in MOAA/S score from pre-dose is considered to indicate the presence of sedation, and such a decrease occurred in a higher number of patients on esketamine than placebo during the short-term Treatment Resistant Depression (TRD) studies. Dose-related increases in the incidence of sedation (MOAA/S score<5) were observed in a fixed-dose TRD study. Table 1 presents the incidence of sedation (MOAA/S score<5) in a fixed-dose study with adult patients<65 years of age with TRD and a flexible-dose study with patients≥65 years of age with TRD.

TABLE 1
Incidence of Sedation (MOAA/S Score <5) in Double-Blind, Randomized,
Placebo-Controlled Studies (Fixed-Dose Study with Adult Patients <65
Years of Age with treatment-resistant depression and Flexible-Dose Study
with Patients ≥65 Years of Age with treatment-resistant depression)

| | Patients <65 years | | | Patients ≥65 years | |
|---|---|---|---|---|---|
| | | SPRAVATO + | | | SPRAVATO + |
| | Placebo + | Oral AD | | Placebo + | Oral AD |
| | Oral AD | 56 mg 84 mg | | Oral AD | 28 to 84 mg |
| | N = 112 | N = 114 | N = 114 | N = 63 | N = 72 |
| Number | 11% | 50% | 61% | 19% | 49% |
| of patients* | | | | | |
| Sedation | | | | | |
| (MOAA/S | | | | | |
| score <5) | | | | | |

*Patients who were evaluated with MOAA/S

In studies for the treatment of depressive symptoms in adults with MDD with acute suicidal ideation or behavior, there was a higher incidence of sedation (MOAA/S score<5) in patients treated with esketamine plus oral antidepressant compared to patients treated with placebo plus oral antidepressant (AD), similar to the treatment-resistant depression study results in Table 1.

### Dissociation/Perceptual Changes

Esketamine can cause dissociative symptoms (including derealization and depersonalization) and perceptual changes (including distortion of time and space, and illusions). In clinical trials, dissociation was transient and occurred on the day of dosing. Dissociation was evaluated by adverse event reports and the Clinician-Administered Dissociative States Scale (CADSS). A CADSS total score of more than 4 indicates the presence of dissociative symptoms, and such an increase to a score of 4 or more occurred in a higher number of patients on esketamine compared to placebo during the short-term treatment-resistant depression studies. Dose-related increases in the incidence of dissociative symptoms (CADSS total score>4 and change>0) were observed in a fixed-dose treatment-resistant depression study. Table 2 presents the incidence of dissociation (CADSS total score>4 and change>0) in a fixed-dose study with adult patients<65 years of age with treatment-resistant depression and a flexible-dose study with patients 65 years of age with treatment-resistant depression.

TABLE 2
Incidence of Dissociation (CADSS Total Score >4 and Change >0) in Double-
Blind, Randomized, Placebo-Controlled Studies (Fixed-Dose Study with Adult
Patients <65 Years of Age with treatment-resistant depression and Flexible-
Dose Study with Patients ≥65 Years of Age with treatment-resistant depression)

| | Patients <65 years | | | Patients ≥65 years | |
|---|---|---|---|---|---|
| | | ESKETAMINE + | | | ESKETAMINE + |
| | Placebo + | Oral AD | | Placebo + | Oral AD |
| | Oral AD | 56 mg | 84 mg | Oral AD | 28 to 84 mg |
| Number of patients* | N = 113 | N = 113 | N = 116 | N = 65 | N = 72 |
| CADSS total score >4 and | 5% | 61% | 69% | 12% | 75% |
| change >0 | | | | | |

*Number of patients who were evaluated with CADSS

In studies for the treatment of depressive symptoms in adults with MDD with acute suicidal ideation or behavior, patients treated with esketamine plus oral AD also demonstrated a higher percentage (84%) of patients with dissociation (CADSS total score>4 and change>0) compared to patients treated with placebo plus oral AD (16%).

Because the subject combination can be administered without dissociation or dissociative events, its risk of abuse is reduced as compared to esketamine. Abuse is the intentional, non-therapeutic use of a drug, even once, for its desirable psychological or physiological effects. Clinical studies with the subject combination of dextromethorphan and bupropion did not reveal drug-seeking behavior, although these observations were not systematic. Therefore, patients with a history of drug abuse may be observed closely for signs of the subject combination of dextromethorphan and bupropion misuse or abuse (e.g., development of tolerance, increases in dose, drug-seeking behavior).

Unlike the combination of quinidine and dextromethorphan, at a dose of a combination of 105 mg of bupropion hydrochloride and 45 mg of dextromethorphan hydrobromide given twice a day, the subject combination does not prolong the QT interval to any clinically relevant extent. Thus, for a human patient who is experiencing major depressive disorder and is at risk of QT prolongation and torsades de pointes, electrocardiographic evaluation of QT interval is not typically conducted on the human patient.

The subject combination may be used for adjunctive treatment of major depressive disorder or depression.

In addition to major depressive disorder, the subject combination may be used to treat other diseases in conditions in the patient populations or circumstances described herein. For example, the subject combination may be used to treat pain or a neurological disorder. Examples of neurological disorders that may be treated with the subject combination include, but are not limited to: affective disorders, psychiatric disorders, cerebral function disorders, movement disorders, dementias, motor neuron diseases, neurodegenerative diseases, seizure disorders, and headaches.

Affective disorders that may be treated by the subject combination include, but are not limited to, depression, major depression, treatment resistant depression, treatment resistant bipolar depression, bipolar disorders including cyclothymia, seasonal affective disorder, mood disorders, chronic depression (dysthymia), psychotic depression, postpartum depression, premenstrual dysphoric disorder (PMDD), situational depression, atypical depression, mania, anxiety disorders, attention deficit disorder (ADD), attention deficit disorder with hyperactivity (ADDH), and attention deficit/hyperactivity disorder (AD/HD), bipolar and manic conditions, obsessive-compulsive disorder, bulimia, obesity or weight-gain, narcolepsy, chronic fatigue syndrome, premenstrual syndrome, substance addiction or abuse, nicotine addiction, psycho-sexual dysfunction, pseudobulbar affect, and emotional lability.

Depression may be manifested by depressive symptoms. These symptoms may include psychological changes such as changes in mood, feelings of intense sadness, despair, mental slowing, loss of concentration, pessimistic worry, agitation, anxiety, irritability, guilt, anger, feelings of worthlessness, reckless behavior, suicidal thoughts, or attempts, and/or self-deprecation. Physical symptoms of depression may include insomnia, anorexia, appetite loss, weight loss, weight gain, decreased energy and libido, fatigue, restlessness, aches, pains, headaches, cramps, digestive issues, and/or abnormal hormonal circadian rhythms.

Psychiatric disorders that may be treated by the subject combination, include, but are not limited to, anxiety disorders, including but not limited to, phobias, generalized anxiety disorder, social anxiety disorder, panic disorder, agoraphobia, obsessive-compulsive disorder, and post-traumatic stress disorder (PTSD); mania, manic depressive illness, hypomania, unipolar depression, depression, stress disorders, somatoform disorders, personality disorders, psychosis, schizophrenia, delusional disorder, schizoaffective disorder, schizotypy, aggression, aggression in Alzheimer's disease, agitation, and agitation in Alzheimer's disease. Alzheimer's disease may also be referred to as dementia of the Alzheimer's type. Other neurobehavioral symptoms of Alzheimer's disease that may be treated include disinhibition and apathy.

Agitation in Alzheimer's disease occurs as the disease progresses. Agitation may present itself as inappropriate verbal, emotional, and/or physical behaviors. Inappropriate behaviors may include, but are not limited to, incoherent babbling, inappropriate emotional response, demands for attention, threats, irritability, frustration, screaming, repetitive questions, mood swings, cursing, abusive language, physical outbursts, emotional distress, restlessness, shredding, sleeping disturbances, delusions, hallucinations, pacing, wandering, searching, rummaging, repetitive body motions, hoarding, shadowing, hitting, scratching, biting, combativeness, hyperactivity, and/or kicking.

Alzheimer's disease (AD) is a progressive neurodegenerative disorder characterized by cognitive decline, and behavioral and psychological symptoms including agitation. AD is the most common form of dementia and afflicts an estimated 6 million individuals in the United States, a number that is anticipated to increase to approximately 14 million by 2050. Agitation is reported in up to 70% of patients with AD and is characterized by emotional distress, aggressive behaviors, disruptive irritability, and disinhibition. Managing agitation is a priority in AD. Agitation in patients with AD has been associated with increased caregiver burden, decreased functioning, accelerated cognitive decline, earlier nursing home placement, and increased mortality. There are currently no therapies approved by the FDA for the treatment of agitation in patients with AD.

Neurobehavioral symptoms have been known to appear during dementia and may be treated by the combination. Caregivers or families may feel more overwhelmed by patients' behavioral/psychological symptoms than by their cognitive impairment. Common forms of the syndrome are Alzheimer's disease, vascular dementia, dementia with Lewy bodies (abnormal aggregates of protein that develop inside nerve cells), and a group of diseases that contribute to frontotemporal dementia (degeneration of the frontal lobe of the brain). The symptoms that dementia patients have are similar to those of psychiatric disorders, but some are slightly different from each other. Neurobehavioral symptoms associated with dementia include depression, apathy, agitation, disinhibition, hallucinations, delusions, psychosis, impulsiveness, aggressiveness, compulsion, excessive sex drive, and personality disorders. Neurobehavioral symptoms such as disinhibition may also be found in other conditions such as traumatic brain injury.

Agitation in patients with Alzheimer's disease may be assessed using the Cohen Mansfield Agitation Inventory or CMAI. The CMAI assesses various behaviors including, Hitting (including self), Kicking, Grabbing onto people, Pushing, Throwing things, Biting, Scratching, Spitting, Hurting self or others, Tearing things or destroying property, Making physical sexual advances, Pacing, aimless wandering, Inappropriate dress or disrobing, Trying to get to a different place, Intentional falling, Eating/drinking inappropriate substances, Handling things inappropriately, Hiding things, Hoarding things, Performing repetitive mannerisms, General restlessness, Screaming, Making verbal sexual advances, Cursing or verbal aggression, Repetitive sentences or questions, Strange noises (weird laughter or crying), Complaining, Negativism, Constant unwarranted request for attention or help.

Schizophrenia may be treated by the combination including positive symptoms and/or negative symptoms of schizophrenia, or residual symptoms of schizophrenia. Other conditions that may treated include intermittent explosive disorder.

Cerebral function disorders that may be treated by the subject combination include, but are not limited to, disorders involving intellectual deficits such as senile dementia, Alzheimer's type dementia, memory loss, amnesia/amnestic syndrome, epilepsy, disturbances of consciousness, coma, lowering of attention, speech disorders, voice spasms, Parkinson's disease, Lennox-Gastaut syndrome, autism, hyperkinetic syndrome, and schizophrenia. Cerebral function disorders also include disorders caused by cerebrovascular diseases including, but not limited to, stroke, cerebral infarction, cerebral bleeding, cerebral arteriosclerosis, cerebral venous thrombosis, head injuries, and the like where symptoms include disturbance of consciousness, senile dementia, coma, lowering of attention, and speech disorders.

Substance addiction abuse that may be treated by the subject combination includes, but is not limited to, drug dependence, addiction to cocaine, psychostimulants (e.g., crack, cocaine, speed, meth), nicotine, alcohol, opioids, anxiolytic and hypnotic drugs, cannabis (marijuana), amphetamines, hallucinogens, phencyclidine, volatile solvents, and volatile nitrites. Nicotine addiction includes nicotine addiction of all known forms, such as smoking cigarettes, cigars and/or pipes, e-cigarettes or vaping, and addiction to chewing tobacco.

Movement disorders that may be treated by the subject combination include, but are not limited to, akathisia, akinesia, associated movements, athetosis, ataxia, ballismus, hemiballismus, bradykinesia, cerebral palsy, chorea, Huntington's disease, Huntington's disease chorea, rheumatic chorea, Sydenham's chorea, dyskinesia, tardive dyskinesia, dystonia, blepharospasm, spasmodic torticollis, dopamine-responsive dystonia, Parkinson's disease, restless legs syndrome (RLS), tremor, essential tremor, and Tourette's syndrome, and Wilson's disease.

Dementias that may be treated by the subject combination include, but are not limited to, Alzheimer's disease, Parkinson's disease, vascular dementia, dementia with Lewy bodies, mixed dementia, fronto-temporal dementia, Creutzfeldt-Jakob disease, normal pressure hydrocephalus, Huntington's disease, Wernicke-Korsakoff Syndrome, and Pick's disease.

Motor neuron diseases that may be treated by the subject combination include, but are not limited to, amyotrophic lateral sclerosis (ALS), progressive bulbar palsy, primary lateral sclerosis (PLS), progressive muscular atrophy, post-polio syndrome (PPS), spinal muscular atrophy (SMA), spinal motor atrophies, Tay-Sach's disease, Sandhoff disease, and hereditary spastic paraplegia.

Neurodegenerative diseases that may be treated the subject combination include, but are not limited to, Alzheimer's disease, prion-related diseases, cerebellar ataxia, spinocerebellar ataxia (SCA), spinal muscular atrophy (SMA), bulbar muscular atrophy, Friedrich's ataxia, Huntington's disease, Lewy body disease, Parkinson's disease, amyotrophic lateral sclerosis (ALS or Lou Gehrig's disease), multiple sclerosis (MS), multiple system atrophy, Shy-Drager syndrome, corticobasal degeneration, progressive supranuclear palsy, Wilson's disease, Menkes disease, adrenoleukodystrophy, cerebral autosomal dominant arteriopathy with subcortical infarcts and leukoencephalopathy (CADASIL), muscular dystrophies, Charcot-Marie-Tooth disease (CMT), familial spastic paraparesis, neurofibromatosis, olivopontine cerebellar atrophy or degeneration, striatonigral degeneration, Guillain-Barre syndrome, and spastic paraplegia.

Seizure disorders that may be treated by the subject combination include, but are not limited to, epileptic seizures, nonepileptic seizures, epilepsy, febrile seizures; partial seizures including, but not limited to, simple partial seizures, Jacksonian seizures, complex partial seizures, and epilepsia partialis continua; generalized seizures including, but not limited to, generalized tonic-clonic seizures, absence seizures, atonic seizures, myoclonic seizures, juvenile myoclonic seizures, and infantile spasms; and status epilepticus.

Types of headaches that may be treated by the subject combination include, but are not limited to, migraine, tension, and cluster headaches.

Other neurological disorders that may be treated by the subject combination include, Rett Syndrome, autism, tinnitus, disturbances of consciousness disorders, sexual dysfunction, intractable coughing, narcolepsy, cataplexy; voice disorders due to uncontrolled laryngeal muscle spasms, including, but not limited to, abductor spasmodic dysphonia, adductor spasmodic dysphonia, muscular tension dysphonia, and vocal tremor; diabetic neuropathy, chemotherapy-induced neurotoxicity, such as methotrexate neurotoxicity; incontinence including, but not limited, stress urinary incontinence, urge urinary incontinence, and fecal incontinence; and erectile dysfunction.

In some embodiments, the subject combination may be used to treat pain, joint pain, pain associated with sickle cell disease, pseudobulbar affect, depression (including treatment resistant depression), disorders related to memory and cognition, schizophrenia, Parkinson's disease, amyotrophic lateral sclerosis (ALS), Rhett's syndrome, seizures, cough (including chronic cough), etc.

In some embodiments, the subject combination may be administered orally to relieve musculoskeletal pain including low back pain, and pain associated with rheumatoid arthritis, juvenile rheumatoid arthritis, osteoarthritis, erosive osteoarthritis, sero-negative (non-rheumatoid) arthropathies, non-articular rheumatism, peri-articular disorders, axial spondyloarthritis including ankylosing spondylitis, Paget's disease, fibrous dysplasia, SAPHO syndrome, transient osteoarthritis of the hip, vertebral crush fractures, osteoporosis, etc.

In some embodiments, the subject combination may be administered to relieve inflammatory pain including musculoskeletal pain, arthritis pain, and complex regional pain syndrome.

Arthritis refers to inflammatory joint diseases that can be associated with pain. Examples of arthritis pain include pain associated with osteoarthritis, erosive osteoarthritis, rheumatoid arthritis, juvenile rheumatoid arthritis, sero-negative (non-rheumatoid) arthropathies, non-articular rheumatism, peri-articular disorders, neuropathic arthropathies including Charcot's foot, axial spondyloarthritis including ankylosing spondylitis, and SAPHO syndrome.

In some embodiments, the subject combination is used to treat chronic musculoskeletal pain.

In some embodiments, the subject composition may be administered to relieve complex regional pain syndrome, such as complex regional pain syndrome type I (CRPS-1), complex regional pain syndrome type II (CRPS-II), CRPS-NOS, or another type of CRPS. CRPS is a type of inflammatory pain. CRPS can also have a neuropathic component. Complex regional pain syndrome is a debilitating pain syndrome. It is characterized by severe pain in a limb that can be accompanied by edema, and autonomic, motor, and sensory changes.

In some embodiments, the subject composition may be administered orally to relieve neuropathic pain.

Examples of neuropathic pain include pain due to diabetic peripheral neuropathy or diabetic peripheral neuropathic pain, post-herpetic neuralgia, trigeminal neuralgia, monoradiculopathies, phantom limb pain, central pain, pain due to multiple sclerosis, etc. Other causes of neuropathic pain include cancer-related pain, lumbar nerve root compression, spinal cord injury, post-stroke pain, central multiple sclerosis pain, HIV-associated neuropathy, and radio- or chemo-therapy associated neuropathy, etc.

In some embodiments, the subject composition may be administered to relieve fibromyalgia.

In some embodiments, the subject composition may be co-administered with one or more strong Inhibitors of CYP2D6. It has been found that the concomitant use of the subject combination with one or more strong CYP2D6 inhibitors increases plasma concentrations of dextromethorphan. It is thus recommended to monitor patients for adverse effects or adverse reactions potentially attributable to dextromethorphan, such as somnolence and dizziness.

The dosage adjustment may be necessary when the subject composition is co-administered with one or more strong inhibitors of CYP2D6. Adjusting to a lower dose amount of the bupropion and/or the dextromethorphan in the subject combination or less frequent dosing of the subject combination may reduce adverse effects or adverse reactions, such as, but not limited to, somnolence, dizziness, or a combination thereof in a patient. For example, a recommended dosage of the subject combination when co-administered with one or more strong CYP2D6 inhibitors, is one tablet containing 45 mg or less of dextromethorphan hydrobromide and 105 mg or less of bupropion hydrochloride once daily, such as once daily in the morning.

Administration of the subject combination once a day to patients who are receiving a concomitant strong CYP2D6 inhibitor may reduce the adverse effects, such as, but not limited to, somnolence, dizziness, or a combination thereof, as compared to administration of the subject combination twice a day for the same number of days. In some embodiments, reducing the dose amount or dose frequency of the subject combination may reduce somnolence. In some embodiments, reducing the dose amount or dose frequency may reduce dizziness. As dizziness may link to falls, adjusting the dosage to lower amount or lower dose frequency of the subject combination may reduce the risk of falls for a patient taking the subject combination. For example, taking the subject combination once a day may reduce the risk of falls for a patient as compared to taking the subject combination twice a day for the same number of days. This may be important, for example, to elderly patients or patients suffering from a dementia, such as Alzheimer's disease.

In some embodiments, the subject composition may be administered to a patient who has moderate renal impairment. As explained herein, it has been found that administering the subject composition to CYP2D6 poor metabolizers increases plasma concentrations of dextromethorphan as compared to patients who are not CYP2D6 poor metabolizers. It is thus recommended to monitor patients for adverse effects or adverse reactions potentially attributable to dextromethorphan, such as somnolence and dizziness.

Dosage adjustment may be necessary when the patient has moderate renal impairment. Adjusting to a lower dose amount of the bupropion and/or the dextromethorphan in the subject combination or less frequent dosing of the subject combination may reduce adverse effects or adverse reactions, or a risk of adverse effects or adverse reactions, such as, but not limited to, somnolence, dizziness, or a combination thereof in a patient. For example, a recommended dosage of the subject combination when administered to a patient who has moderate renal impairment, is one tablet containing 45 mg or less of dextromethorphan hydrobromide and 105 mg or less of bupropion hydrochloride once daily, such as once daily in the morning.

Administration of the subject combination once a day to patients who have moderate renal impairment may reduce the adverse effects, or the risk of adverse effects, such as, but not limited to, somnolence, dizziness, or a combination thereof, as compared to administration of the subject combination twice a day for same number of days. In some embodiments, reducing the dose amount or dose frequency of the subject combination may reduce somnolence. In some embodiments, reducing dose amount or dose frequency may reduce dizziness. As dizziness may link to falls, adjusting the dosage to lower amount or lower dose frequency of the subject combination may reduce the risk of falls for a patient taking the subject combination. For example, taking the subject combination once a day may reduce the risk of falls for a patient as compared to taking the subject combination twice a day for same number of days. This may be important, for example, to elderly patients or patients suffering from a dementia, such as Alzheimer's disease.

The term “treating” or “treatment” includes the diagnosis, cure, mitigation, treatment, or prevention of disease in man or other animals, or any activity that otherwise affects the structure or any function of the body of man or other animals.

A subject combination may be used to treat any disease or condition identified as treatable by the combination of bupropion and dextromethorphan in any of the following U.S. Pat. Nos. 8,569,328, 9,168,234, 9,189,905 9,205,083, 9,238,032, 9,278,095, 9,314,462, 9,370,513, 9,375,429, 9,408,815, 9,421,176, 9,457,023, 9,457,025, 9,474,731, 9,486,450, 9,700,528, 9,700,553, 9,707,191, 9,763,932, 9,861,595, 9,867,819, 9,968,568, 10,058,518, 10,064,857, 10,080,727, 10,092,560, 10,092,561, 10,105,327, 10,105,361, 10,251,879, 10,463,634, 10,512,643, 10,548,857, 10,596,167, 10,772,850, 10,780,064, 10,780,066, 10,786,469, 10,786,496, 10,799,497, 10,806,710, 10,864,209, 10,874,663, 10,874,664, 10,874,665, 10,881,624, 10,881,657, 10,894,046, 10,894,047, 10,898,453, all of which are incorporated by reference herein in their entireties for their disclosure of diseases that may be treated by a combination of bupropion and dextromethorphan, including specific embodiments and combinations described therein.

### Example 1

In a study of the subject combination in 7 subjects with moderate (GFR 30-60 mL/min) renal impairment compared to 6 matched controls with normal renal function (matched in gender, age, and weight range to impaired subjects), both dextromethorphan and bupropion exposures increased by approximately 2-fold and clearances were reduced by 50%.

### Example 2

Approximately 7% to 10% of Caucasians and 3% to 8% of African Americans lack the capacity to metabolize CYP2D6 substrates and are classified as poor metabolizers. In 3 poor metabolizers the pharmacokinetics of the subject combination resulted in an approximate 3-fold and 3.4-fold increase in dextromethorphan Cₘₐₓ and AUC₀₋₁₂, respectively, compared to extensive metabolizers. An exploration of steady state pharmacokinetic data in 12 poor metabolizers treated with the subject combination in efficacy trials showed plasma concentrations of dextromethorphan that were generally higher than exposures for non-poor metabolizers.

### Example 3

Co-administration of the SSRI paroxetine and the subject combination comprising dextromethorphan and bupropion was studied in 29 healthy volunteers. Paroxetine increased the overall exposure of dextromethorphan by 2.5-fold and had no effect on bupropion. The overall exposure of paroxetine was increased by 1.2-fold when co-administered with the subject combination. Based on these results, when the subject combination is prescribed with drugs that inhibit CYP2D6, the subject combination should be dosed once daily. Use caution when administering the subject combination in conjunction with drugs which are extensively metabolized via CYP2D6.

### Example 4

Tablets are prepared for oral administration. They are round bilayer tablets. Each tablet contains 45 mg dextromethorphan hydrobromide (equivalent to 32.98 mg of the dextromethorphan free base) in an immediate-release formulation and 105 mg bupropion hydrochloride (equivalent to 91.14 mg of the bupropion free base) in an extended-release formulation. Each tablet contains the following inactive ingredients: carbomer homopolymer, colloidal silicon dioxide, crospovidone, glyceryl monocaprylocaprate, L-cysteine hydrochloride monohydrate, magnesium stearate, microcrystalline cellulose, polyvinyl alcohol, red iron oxide, sodium lauryl sulfate, stearic acid, talc, titanium dioxide, and/or yellow iron oxide.

### Example 5

The properties of the tablets of Example 4, containing a combination of dextromethorphan hydrobromide, which is an uncompetitive NMDA receptor antagonist and sigma-1 receptor agonist, and bupropion hydrochloride, which is an aminoketone and CYP450 2D6 inhibitor, were studied.

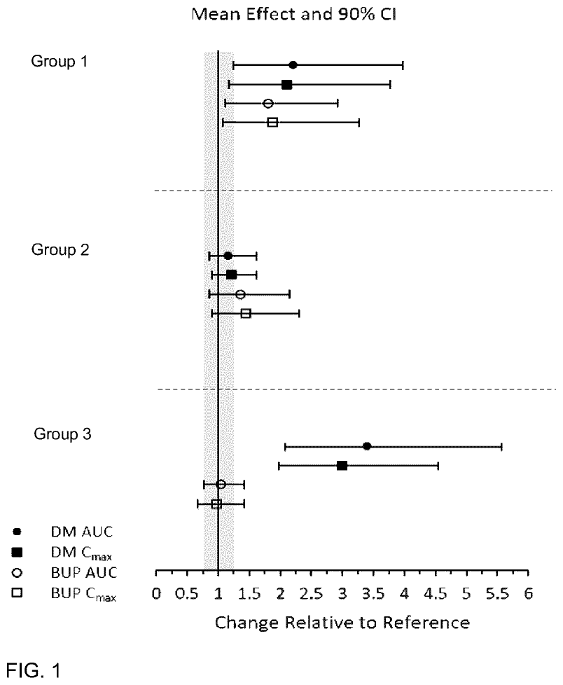

The effects of renal impairment, hepatic impairment, and CYP2D6 poor metabolizer status on the exposure to the tablets of Example 4 are summarized in FIG. 1.

Results depicted in FIG. 1 are based on plasma concentrations in human patients after 8 days of twice daily dosing of a tablet containing 45 mg of dextromethorphan hydrobromide and 105 mg of bupropion hydrochloride. Data are GMRs and 90% CIs. References used are the matched healthy subjects for renal and hepatic impairment studies, and extensive or ultra-extensive CYP2D6 metabolizers respectively. AUC represents the area under the plasma concentration-time curve from zero to 12 hours; BUP represents bupropion; Cl is confidence interval; Cₘₐₓ is maximum plasma concentration; DM represents dextromethorphan; GMRs represents geometric mean ratios; PK represents pharmacokinetics.

In FIG. 1, the results for patients with moderate Renal Impairment are identified as “Group 1,” the results for patients with Moderate Hepatic Impairment are identified as “Group 2,” and the results for patients who are CYP2D6 Poor Metabolizers are identified as “Group 3.” The results are summarized in Table 3 below.

TABLE 3

| | | | Change Relative |
|---|---|---|---|
| | | PK | to Reference |
| Patients | Group | Parameter | (90% CI) |
| Moderate Renal Impairment | 1 | DM AUC | 2.21 (1.24, 3.97) |
| Moderate Renal Impairment | 1 | DM Cₘₐₓ | 2.10 (1.17, 3.77) |
| Moderate Renal Impairment | 1 | BUP AUC | 1.80 (1.12, 2.92) |
| Moderate Renal Impairment | 1 | BUP Cₘₐₓ | 1.87 (1.07, 3.27) |
| Moderate Hepatic | 2 | DM AUC | 1.17 (0.85, 1.61) |
| Impairment | | | |
| Moderate Hepatic | 2 | DM Cₘₐₓ | 1.21 (0.90, 1.62) |
| Impairment | | | |
| Moderate Hepatic | 2 | BUP AUC | 1.36 (0.86, 2.15) |
| Impairment | | | |
| Moderate Hepatic | 2 | BUP Cₘₐₓ | 1.44 (0.90, 2.30) |
| Impairment | | | |
| CYP2D6 Poor Metabolizer | 3 | DM AUC | 3.40 (2.08, 5.57) |
| CYP2D6 Poor Metabolizer | 3 | DM Cₘₐₓ | 3.00 (1.98, 4.54) |
| CYP2D6 Poor Metabolizer | 3 | BUP AUC | 1.04 (0.77, 1.41) |
| CYP2D6 Poor Metabolizer | 3 | BUP Cₘₐₓ | 0.97 (0.67, 1.41) |

For example, for patients having moderate hepatic impairment (Group 2), the AUC₀₋₁₂ and Cₘₐₓ of dextromethorphan and bupropion were not significantly different from that for healthy patients.

The mean ratio of AUC₀₋₁₂ of dextromethorphan for patients with moderate hepatic impairment as compared to healthy patients was 1.17, with a 90% confidence interval of 0.85-1.61.

The mean ratio of Cₘₐₓ of dextromethorphan for patients with moderate hepatic impairment as compared to healthy patients was 1.21, with a 90% confidence interval of 0.90-1.62.

The mean ratio of AUC₀₋₁₂ of bupropion for patients with moderate hepatic impairment as compared to healthy patients was 1.36, with a 90% confidence interval of 0.86-2.15.

The mean ratio of Cₘₐₓ of bupropion for patients with moderate hepatic impairment as compared to healthy patients was 1.44, with a 90% confidence interval of 0.90-2.30.

Based upon these results, no dosage adjustment is recommended in patients known to have mild or moderate hepatic impairment.

For patients having moderate renal impairment, a 2.21-fold increase in dextromethorphan AUC₀₋₁₂, a 2.10-fold increase in dextromethorphan Cₘₐₓ, a 1.80-fold increase in bupropion AUC₀₋₁₂, and a 1.87-fold increase in bupropion Cₘₐₓ were observed.

Based upon these results, dosage adjustment is recommended in patients known to have moderate renal impairment because these patients have higher dextromethorphan and bupropion concentrations than patients with healthy renal function. The recommended total daily dose for patients known to have moderate renal impairment is about 45 mg of dextromethorphan hydrobromide and about 105 mg of bupropion hydrochloride (e.g. one tablet containing about 45 mg of dextromethorphan hydrobromide and about 105 mg of bupropion hydrochloride for administration once daily, such as in the morning), or an equivalent dose of another form dextromethorphan and/or bupropion.

### Example 6

The effect of concomitantly administered 20 mg of paroxetine on the dextromethorphan exposure to patients taking, twice daily, the tablet of Example 4 containing 45 mg of dextromethorphan hydrobromide and 105 mg of bupropion hydrochloride was determined. Dextromethorphan AUC₀₋₁₂ was increased 2.69-fold and dextromethorphan Cₘₐₓ was increased 2.38-fold as compared to patients taking the tablet twice daily without paroxetine.

### Example 7

The efficacy of the tablets of Example 4 for the treatment of major depressive disorder in adults was demonstrated in a placebo-controlled clinical study (Study 1). In this study, adult patients (18 to 65 years of age) who met the Diagnostic and Statistical Manual of Mental Disorders (DSM-5) criteria for MDD were randomized to receive the tablets of Example 4 (“DM/BU,” 45 mg of dextromethorphan hydrobromide and 105 mg of bupropion hydrochloride) twice daily (N=156) or placebo twice daily (N=162) for 6 weeks. Patients in Study 1 had a median age of 41 years and were 67% female, 55% Caucasian, 35% Black, and 5% Asian. Here, “N” represents number of adult patients.

The primary outcome measure was the change from baseline to Week 6 in the total score of the Montgomery-Asberg Depression Rating Scale (MADRS). The MADRS is a clinician-rated scale used to assess the severity of depressive symptoms. Patients are rated on 10 items to assess feelings of sadness, inner tension, reduced sleep or appetite, difficulty concentrating, lassitude, lack of interest, pessimism, and suicidality. Scores on the MADRS range from 0 to 60, with higher scores indicating more severe depression. DM/BU was statistically significantly superior to placebo in improvement of depressive symptoms as measured by decrease in MADRS total score at Week 6 (see Table 4).

TABLE 4
Primary Efficacy Results for Change from Baseline in MADRS
Total Score at Week 6 in Adult Patients with MDD

| | | Mean | LS Mean | LS Mean |
|---|---|---|---|---|
| | Treatment | Baseline | Change from | Difference^{a} |
| Study | Group | Score (SD) | Baseline (SE) | (95% CI) |
| Study 1 | DM/BU | 33.6 (4.4) | −15.9 (0.9) | −3.9 |
| | (N = 156) | | | (−6.4, −1.4) |
| | Placebo (N = 162) | 33.2 (4.4) | −12.1 (0.9) | — |

SD = standard deviation; SE = standard error; LS Mean = least-squares mean; CI = confidence interval.
^{a}drug-placebo

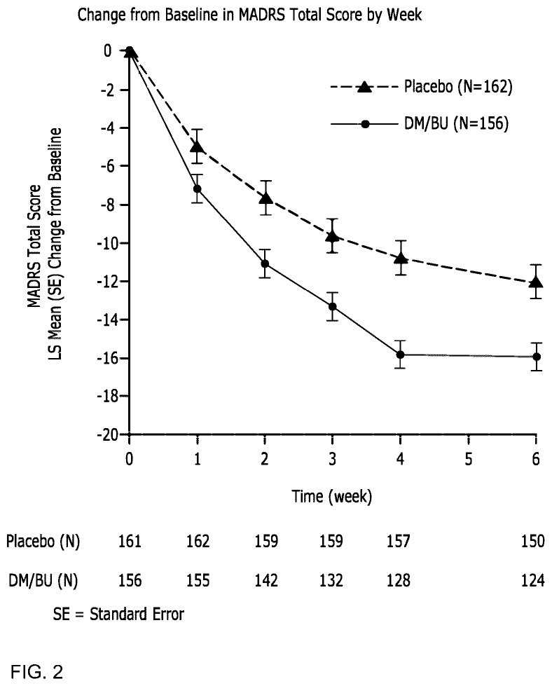

The change from baseline in MADRS total score by week is displayed in FIG. 2. The change in MADRS total score from baseline to Week 1 and from baseline to Week 2 were pre-specified secondary efficacy endpoints. The difference between DM/BU and placebo in change from baseline in MADRS total score was statistically significant at Week 1 and at Week 2. ^{a} drug—placebo

Examination of demographic subgroups by age, sex, and race did not suggest differences in response.

Unless otherwise indicated, all numbers expressing quantities of ingredients, properties such as amounts, percentage, and so forth used in the specification and claims are to be understood in all instances as indicating both the exact values as shown and as being modified by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

Use of the term “comprising” or “comprises” herein also contemplates that use of “consisting essentially of,” “consists essentially of,” “consisting of,” or “consists of” in its place.

Affirmative recitation of an element anywhere herein should be understood to contemplate both including and excluding that element.

The terms “a,” “an,” “the” and similar referents used in the context of describing the embodiments (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the embodiments and does not pose a limitation on the scope of any claim. No language in the specification should be construed as indicating any non-claimed element essential to the practice of the claims.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from a group, for reasons of convenience and/or to expedite prosecution. When any such inclusion or deletion occurs, the specification is deemed to contain the group as modified thus fulfilling the written description of all Markush groups if used in the appended claims.

Certain embodiments are described herein, including the best mode known to the inventors for carrying out the claimed embodiments. Of course, variations on these described embodiments will become apparent to those of ordinary skill in the art upon reading the foregoing description. The inventor expects skilled artisans to employ such variations as appropriate, and the inventors intend for the claimed embodiments to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A method of treating a neurological disorder, comprising administering a bupropion, or a combination of a bupropion and a dextromethorphan, to a human being in need thereof, monitoring the human being for Brugada pattern/syndrome, subcutaneous tissue disorder, drug reaction with eosinophilia and systemic symptoms, or acute generalized exanthematous pustulosis, and reducing or discontinuing use of the bupropion or the combination of the bupropion and the dextromethorphan if the human being develops Brugada pattern/syndrome, subcutaneous tissue disorder, drug reaction with eosinophilia and systemic symptoms (DRESS), or acute generalized exanthematous pustulosis.

2. The method of claim 1, wherein the human being develops subcutaneous tissue disorder.

3. The method of claim 1, wherein the human being develops DRESS.

4. The method of claim 1, wherein the human being develops Brugada pattern/syndrome.

5. The method of claim 1, wherein the human being develops acute generalized exanthematous pustulosis.

6. The method of claim 1, wherein the bupropion is bupropion hydrochloride, or a molar equivalent amount of the free base form or another salt form of bupropion.

7. The method of claim 1, wherein the dextromethorphan is dextromethorphan hydrobromide, or a molar equivalent amount of the free base form or another salt form of dextromethorphan.

8. The method of claim 1, wherein the bupropion is in sustained release formulation.

9. The method of claim 1, wherein the dextromethorphan is in immediate release formulation.

10. The method of claim 1, wherein the bupropion or the combination of the bupropion and the dextromethorphan is in a dosage form.

11. The method of claim 10, wherein the dosage form further comprises: colloidal silicon dioxide, crospovidone, glyceryl monocaprylocaprate, L-cysteine hydrochloride monohydrate, magnesium stearate, microcrystalline cellulose, polyvinyl alcohol, red iron oxide, sodium lauryl sulfate, stearic acid, talc, titanium dioxide, or yellow iron oxide, or a combination thereof.

12. The method of claim 11, wherein the dosage form is an oral dosage form.

13. The method of claim 12, wherein the oral dosage form is a tablet.

14. The method of claim 13, wherein the tablet is a bilayer tablet.

15. The method of claim 1, wherein the combination of the bupropion and the dextromethorphan is administered to the human being.

16. The method of claim 1, wherein a combination of: 1) about 100-110 mg, about 104-106 mg, or about 105 mg of bupropion hydrochloride, or a molar equivalent amount of the free base form or another salt form of bupropion; and 2) about 40-50 mg, about 44-46 mg, or about 45 mg of dextromethorphan hydrobromide, or a molar equivalent amount of the free base form or another salt form of dextromethorphan, is administered once a day or twice a day to the human being.

17. The method of claim 1, wherein about 105 mg or less per day of the bupropion is administered after the human being develops Brugada pattern/syndrome, subcutaneous tissue disorder, DRESS, or acute generalized exanthematous pustulosis.

18. The method of claim 1, wherein a combination comprising about 105 mg or less of bupropion hydrochloride and about 45 mg or less of dextromethorphan hydrobromide is administered per day after the human being develops Brugada pattern/syndrome, subcutaneous tissue disorder, DRESS, or acute generalized exanthematous pustulosis.

19. The method of claim 1, wherein administration of the bupropion is discontinued after the human being develops Brugada pattern/syndrome, subcutaneous tissue disorder, DRESS, or acute generalized exanthematous pustulosis.

20. The method of claim 1, wherein administration of the combination of the bupropion and the dextromethorphan is discontinued after the human being develops Brugada pattern/syndrome, subcutaneous tissue disorder, DRESS, or acute generalized exanthematous pustulosis.
